(19) United States
(12) Patent Application Publication
(10) Pub. No.: US 2015/0224270 A1
(43) Pub. Date: Aug. 13, 2015

(54) RESISTANCE BREATHING DEVICE

(71) Applicant: JUSTIN FRANDSON, Newport Beach, CA (US)

(72) Inventor: JUSTIN FRANDSON, NEWPORT BEACH, CA (US)

(21) Appl. No.: 14/618,843

(22) Filed: Feb. 10, 2015

Related U.S. Application Data
(60) Provisional application No. 61/938,992, filed on Feb. 12, 2014.

(51) Int. Cl. A61M 16/00; A61M 16/04

(52) U.S. Cl. CPC A61M 16/0003; A61M 16/049; A61M 2205/586

(57) ABSTRACT

A resistance breathing device having a pair of coaxially aligned cup members, which collectively define an inner chamber and an outer annular chamber surrounding the inner chamber. The inner cup member includes an end wall submerged within water located within the outer cup member, wherein the end wall includes a plurality of openings therein. A lid extends over the cup members and defines a plurality of openings in communication with the annular chamber. A mouthpiece is coupled to the lid and allows a user to breathe in and out through the device for resistance breathing training.

### CROSS-REFERENCE TO RELATED APPLICATIONS

This application claims the benefit of U.S. Provisional Application No. 61/938,992, filed Feb. 12, 2014, the contents of which are expressly incorporated herein by reference.

### STATEMENT RE: FEDERALLY SPONSORED RESEARCH/DEVELOPMENT

(Not Applicable)

### BACKGROUND OF THE INVENTION

The present invention relates generally to a breathing trainer, and more specifically to a resistance breathing device.

Breathing is an essential human activity. In particular, it is the process which moves air in and out of the lungs as a means of providing oxygen to the body, and also as a means of expelling carbon dioxide from the body. The process of moving air into the lungs is generally referred to as inhalation, while the process of moving air out of the lungs is generally referred to as exhalation.

It is believed to two essential factors in maintaining one's health are oxygen and water. Along these lines, many harmful viruses thrive in acidic and anaerobic (e.g., little or no oxygen) environments. Viruses may be combated by enhancing an alkaline intake, as well as oxygen. Enhancing one's ability to breathe more effectively may improve oxygenation of the body, which in turn, promotes a healthier environment. An enhanced ability to breathe may be achieved through regular breathing training, which may also be associated with mood improvement, sleep improvement, a strengthened resistance to disease or illness, and improved physical and mental activity.

The importance of breathing training tends to have increased importance for athletes due to the increased load placed on the body, which requires additional oxygen. Along these lines, the body employs respiratory muscles in order to accommodate the increased demands placed on a typical athlete's respiratory system. Individuals with weaker inspiratory muscles may experience periods of breathlessness, particularly during abnormally strenuous activities or in the case of elderly individuals. Therefore, resistance breathing training is believed to strengthen the inspiratory muscles

As can be seen, there exists a need in the art for an improved breathing/training apparatus.

### BRIEF SUMMARY OF THE INVENTION

The above-mentioned deficiencies and needs associated with flow control elements of the prior art are specifically addressed and alleviated by various aspects of the invention as disclosed herein.

According to one aspect of the invention, there is provided a resistance breathing device including a first body having a first internal chamber, wherein the first body defines a primary opening. The first body further includes a secondary opening in fluid communication with the first internal chamber, and a breathing opening spaced from the primary and secondary openings and in fluid communication with the first internal chamber. The device further includes an outer cup member having an open end portion and an opposed closed end portion, wherein the outer cup member is sized and configured to be operatively connectable to the first body. A portion of the first body is received within the outer cup member when the first body is connected to the outer cup member to define a second internal chamber between the first body and the outer cup member. The second internal chamber is fluidly connectable to the first internal chamber via the secondary opening. The outer cup member is configured to receive a liquid therein such that the secondary opening is submerged in the liquid to provide resistance to gas flowing between the first and second internal chambers.

The first body may include an inner cup member having a closed end portion and an opposed open end portion, and a lid connectable to the open end portion of the inner cup member. The primary opening and the breathing opening may be formed in the lid and the secondary opening may be formed in the closed end portion of the inner cup member. The outer cup may define an inner diameter of approximately 2.75 inches and an internal length of approximately 5 inches. The inner cup member may define an outer diameter of approximately 2 inches and an external length of approximately 4.875 inches.

The breathing device may be configured such that the second internal chamber circumscribes the first internal chamber.

The outer cup member may include measurement lines formed on an inner surface thereof to denote liquid levels associated with respective levels of resistance.

The resistance breathing device may further include a handle coupled to the outer cup member to facilitate holding/gripping of the device.

The resistance breathing device may additionally comprise a straw connectable to the first body to fluidly connect the straw to the breathing opening. A mouthpiece may be integral with the straw to facilitate breathing through the straw.

The primary opening may include a plurality of primary holes and the secondary opening may include a plurality of secondary holes. The plurality of secondary holes may define at least two different shapes.

The first body and the cup member may be formed from a dishwasher safe material.

The resistance breathing device may additionally include a metering device coupled to the first body and moveable relative thereto for selectively opening and closing the secondary opening. The metering device may be rotatable relative to the first body.

According to another aspect of the present invention, there is provided a resistance breathing device including a main body having a first gas port and a second gas port spaced from the first gas port. The main body defines a gas transfer passageway between the first and second gas ports. The main body further includes a reservoir within the gas transfer passageway, wherein the reservoir is fillable with a liquid to provide resistance to gas transfer between the first and second gas ports. The main body defines a longitudinal axis and is of an axial length that is approximately 5 inches.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a side sectional view of a resistance breathing device constructed in accordance with an embodiment of the present invention;

FIG. 2 is an exploded upper perspective view of the resistance breathing device;

FIG. 3 is an exploded lower perspective view of the resistance breathing device;

FIG. 4 is a top view of a lid having a plurality of openings formed therein; and

FIG. 5 is a bottom view of an inner cup member having a plurality of openings formed in a closed end portion thereof.

### DETAILED DESCRIPTION OF THE INVENTION

The detailed description set forth below in connection with the appended drawings is intended as a description of certain embodiments of a resistance breathing training device and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual relationship or order between such entities.

Referring now to the drawings wherein the showings are for purposes of illustrating preferred embodiments of the present invention and not for purposes of limiting the same, there is depicted an embodiment of a resistance breathing device 10 configured for resistance breathing training exercises, which enhances a user's health, wellness, anti-aging, injury recovery (e.g., concussions) and performance needs. The unique and innovative design of the resistance breathing device 10 allows for ease of use, cleaning, and transport of the device 10.

According to one embodiment, the resistance breathing device 10 generally includes an outer cup member 12, an inner cup member 14, a lid 16, and a mouthpiece 18.

The outer cup member 12 includes an open end portion 20 (e.g., the upper end) and an opposed closed end portion 22 (e.g., the lower end). The outer cup member 12 defines an outer cup side wall 24 extending between the open end portion 20 and the closed end portion 22, with the outer cup side wall 24 having an outer surface 26 and an opposed inner surface 28 which defines an outer cup chamber 30. The outer cup member 12 may include measurement lines formed on the side wall inner surface thereof to denote liquid levels associated with respective levels of resistance, as will be described in more detail below. The outer cup side wall 24 terminates at the open end portion 20 to define an outer cup member rim 32. An outer cup end wall 34 is coupled to the outer cup side wall 24 at the closed end portion 22, with the outer cup end wall 34 defining an outer surface 36 and an opposing inner surface 38 which partially defines the outer cup chamber 12.

The inner surface 28 of the outer cup member side wall 24 defines an inner diameter. Furthermore, the outer cup member rim 32 and the inner surface 38 of the end wall 34 define an internal length. According to one embodiment, the outer cup member 12 defines an inner diameter of approximately 2.75 inches and an internal length of approximately 5 inches. However, those skilled in the art will readily appreciate that the inner diameter and the internal length may be varied without departing from the spirit and scope of the present invention.

In the exemplary embodiment, the outer cup member 12 defines a circular, cylindrical configuration, although it is understood that the configuration of the outer cup member 12 is not limited thereto. For instance, the outer cup member 12 may define a quadrangular configuration, triangular configuration or any other configuration known in the art without departing from the spirit and scope of the present invention.

The inner cup member 14 includes an open end portion 40 (e.g., the upper end) and an opposed closed end portion 42 (e.g., the lower end). The inner cup member 14 defines an inner cup side wall 44 extending between the open end portion 40 and the closed end portion 42, with the inner cup side wall 44 having an outer surface 46 and an opposed inner surface 48 which defines an inner cup chamber 50. When the resistance breathing device 10 is assembled, the inner cup 14 is placed within the outer cup 12, with the inner surface 28 of the outer cup side wall 24 being spaced from the outer surface 46 of the inner cup side wall 44 so as to define an annular chamber 52 therebetween. In this respect, the annular chamber 52 and the inner cup chamber 50 both comprise portions of the outer cup chamber 30 when the inner cup member 14 is placed within the outer cup member 12.

The inner cup side wall 44 terminates at the open end portion 40 to define an inner cup member rim 54. An inner cup end wall 56 is coupled to the inner cup side wall 44 at the closed end portion 42, with the inner cup end wall 56 defining an outer surface 58 and an opposing inner surface 60 which partially defines the inner cup chamber 50. FIGS. 3 and 5 show an embodiment of the inner cup end wall 56 having a plurality of openings formed therein, the purpose of which will be described in more detail below. The openings are arranged in three distinct annular regions, with the openings in a common annular region being of similar shape, which differs from the openings on the other annular regions. In particular, an inner annular region includes four triangular-shaped openings 62, a middle annular region includes eight square-shaped openings 64, while an outer annular region includes twelve circular openings 66. Those skilled in the art will readily appreciate that the size, shape and number of openings formed on the inner cup end wall 56 may be varied without departing from the spirit and scope of the present invention. The different shape openings allow for a different resistance, as can be determined by one of ordinary skill in the art.

An inner cup metering device (not shown) may be coupled to the inner cup member 14 and moveable relative thereto for selectively opening and closing the openings formed in the inner cup end wall 56. According to one embodiment, the inner cup metering device is a disk which rotates relative to the inner cup member 14, wherein the disk includes a plurality of disk openings formed therein. The inner cup metering device may be transitional between an open configuration and a closed configuration. In the open configuration, the disk openings are aligned with the inner cup end wall openings, and in the closed configuration, the disk openings are moved out of alignment with the inner cup end wall openings via rotation of the metering device to either completely or substantially block the inner cup end wall openings.

The inner surface 48 of the inner cup member side wall 44 defines an inner diameter. Furthermore, the inner cup member rim 54 and the outer surface 58 of the end wall 56 define an external length. According to one embodiment, the inner cup member 14 defines an inner diameter of approximately 2 inches and an external length of approximately 4.875 inches. However, those skilled in the art will readily appreciate that the inner diameter and the internal length may be varied without departing from the spirit and scope of the present invention.

In the exemplary embodiment, the inner cup member 14 defines a circular, cylindrical configuration, although it is understood that the configuration of the outer cup member 14 is not limited thereto. For instance, the inner cup member 14 may define a quadrangular configuration, triangular configuration or any other configuration known in the art without departing from the spirit and scope of the present invention. It may be desirable to configure the outer and inner cup members 12, 14 to be of a similar configuration (e.g., both circular cylindrical), although those skilled in the art will recognize that similarly configured cup members 12, 14 are not required.

The lid 16 includes a lid wall 68 having a first surface 70 (e.g., a top surface) and an opposing second surface 72 (e.g., a bottom surface). A pair of collars 74, 76 extend downwardly from the second surface 72, with the outermost collar 74 being adapted to interface with the outer cup member 12 and the innermost collar 76 being adapted to interface with the inner cup member 14. In the exemplary embodiment, the outermost collar 74 and the innermost collar 76 are coaxially aligned with each other, with the outermost collar 74 extending around the outer surface 26 of the outer cup member 12, and the innermost collar 76 extending around the outer surface 46 of the inner cup member 14. The lid 16 additionally includes an upwardly extending neck 78 defining an opening 80 which is in fluid communication with the inner chamber 50 when the lid 16 is coupled to the inner cup member 14, as will be described in more detail below.

The lid 16 additionally includes a plurality of lid openings 82 extending through the lid wall 68 to allow for fluid flow therethrough. In the exemplary embodiment, the lid openings 82 are formed adjacent an outer periphery of the lid 16, such that when the lid 16 is coupled to the outer and inner cup members 12, 14, the openings 82 are positioned between the outer and inner cup members 12, 14 (e.g., the openings 82 are in fluid communication with the annular chamber 52). In the exemplary embodiment, the lid 16 includes eighteen circular lid openings 82, although it is understood that the number and shape of the lid openings 82 may vary without departing from the spirit and scope of the present invention.

The mouthpiece 18 is a straw-like member which defines a fluid pathway 84 between the lid neck 78 and the user's mouth. Along these lines, the mouthpiece 18 includes a first end portion 86 connectable to the lid neck 78 and a second end portion 88 adapted to interface with the user's mouth. The first end portion 86 is configured to engage with the lid neck 78 to physically connect the mouthpiece 18 to the lid 16, while also creating a fluid interconnection therebetween, such that when a user breathes in through the mouthpiece 18, a vacuum or negative pressure is created in the inner cup chamber 50 and when a user breathes out through the mouthpiece 18, a positive pressure is created in the inner cup chamber 50. The mouthpiece 18 may include an elbow or bent section 90 located between the first and second end portions 86, 88, which may allow the user to more easily position the second end portion 88 adjacent the user's mouth. The mouthpiece 18 may be fabricated from suitable plastic materials known in the art, which is durable and able to last several years. The mouthpiece 18 may be of a prescribed height which allows for proper posture when using the device 10, as well as a farther distance for the air to travel, which enhances the overall resistance. According to one embodiment, the mouthpiece 18 is approximately 10″ long and the bend in the upper ¾ preferably does not have groves to bend the entire way. This will allow durability of the product for many years.

According to one embodiment, the device 10 includes a handle 92 coupled to, and preferably integrally formed with, the outer cup member 12 to facilitate holding of the device 10, although it is understood that the handle 92 is optional, and thus, other embodiments may be formed without a handle 92.

In order to facilitate cleansing of the device 10, it is preferred that the various components of the device 10 are preferably formed of a dishwasher safe material.

With the basic structural components of the resistant breathing device 10 described above, the following discussion will focus on an exemplary usage of the device 10. The device 10 is used by adding water inside the outer cup member 12. Approximately ½″ of water may be added to the outer cup member 12, although more or less water may be used depending on the desired resistance. Furthermore, although water is used in the exemplary embodiment, it is understood that other fluids may also be used. The inner cup member 14 is then placed within the outer cup member 12, with the end wall 56 of the inner cup member 14 being submerged within the water. The lid 16 is then placed over the outer and inner cup member 12, 14 and the mouthpiece 18 is engaged with the lid 16. If the device 10 includes one or more metering devices, the user may adjust the metering devices to a desired position.

When the device 10 is ready for use, the user may sit or stand, while placing his/her mouth in contact with the mouthpiece 18. The user's sternum should be up and the torso lengthened, e.g., as though a string was drawing the user up from the crown of his/her head. The user keeps the chin in and breathes in and out of the mouthpiece 18.

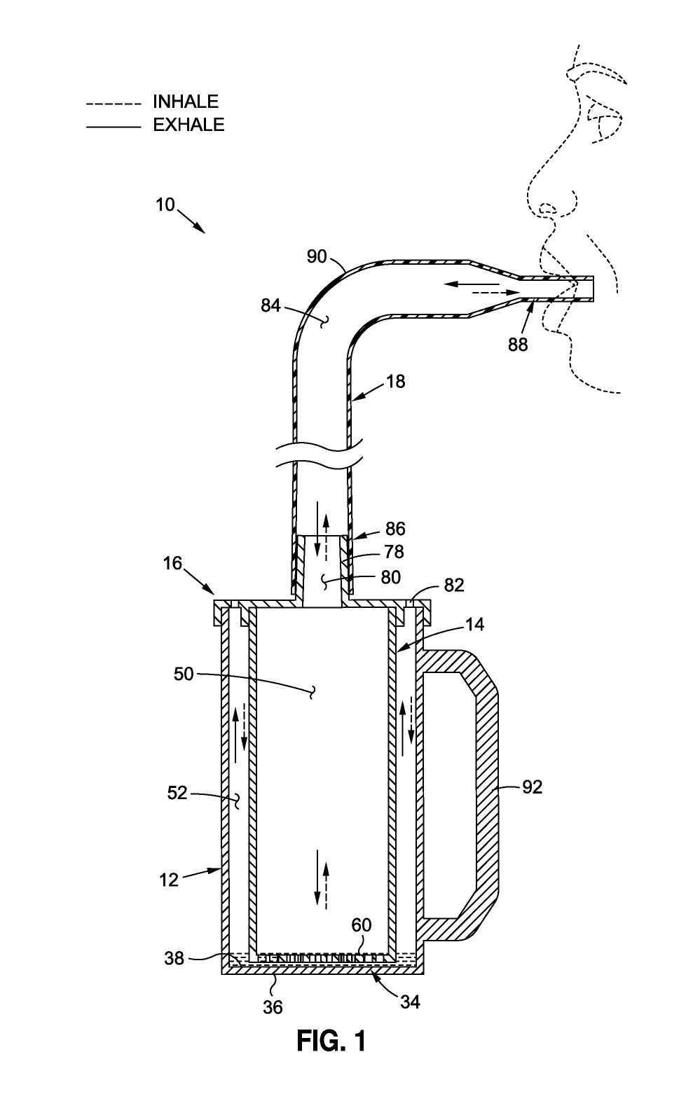

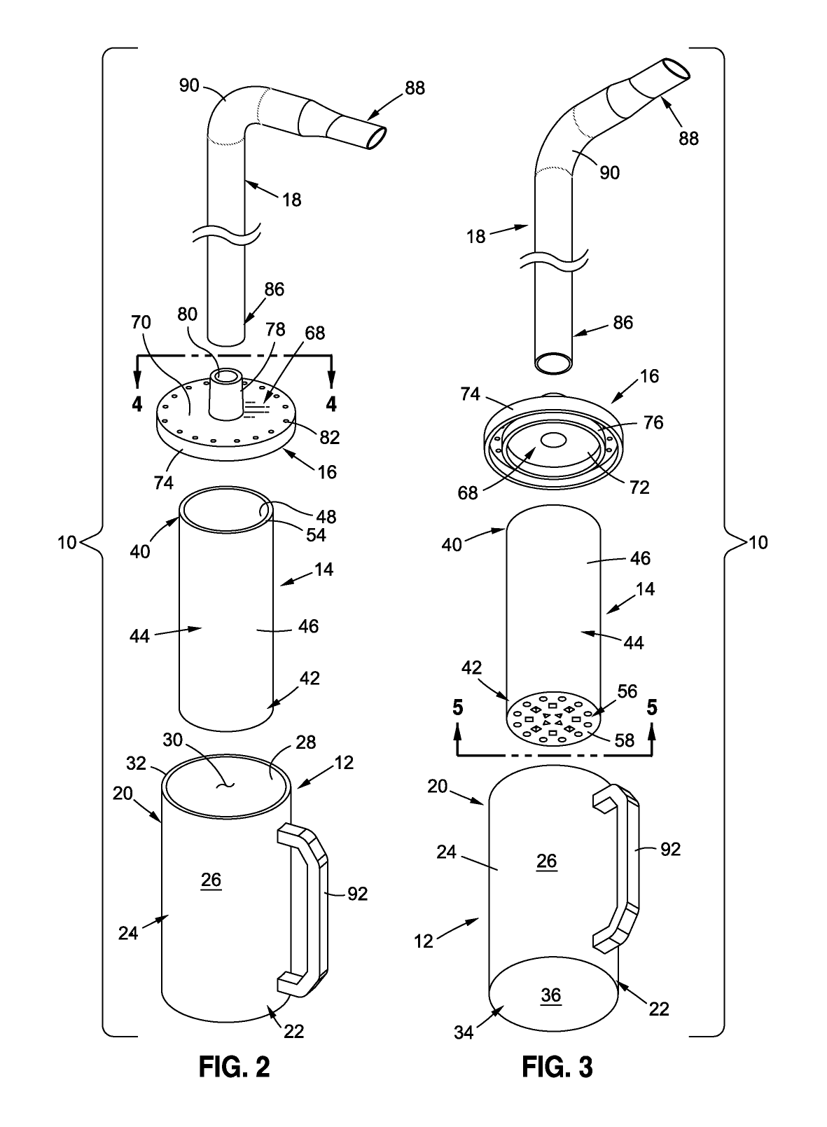

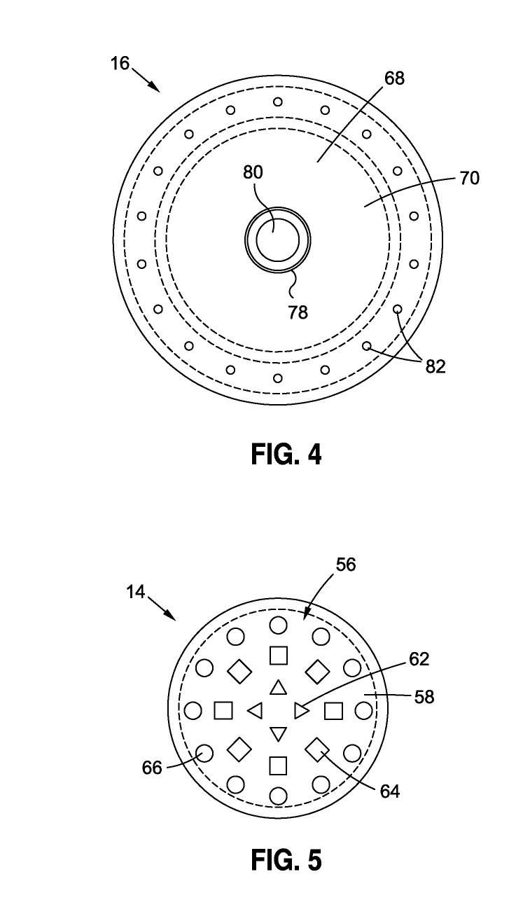

When the user breathes in, the airflow through the device 10 is represented by the dashed arrows shown in FIG. 1, while the solid arrows represent the airflow when the user breathes out. When the user breathes in, the user creates a vacuum or negative pressure within the mouthpiece 18 and the inner chamber 50. When the magnitude of the negative pressure within the inner chamber 50 is large enough, air is transferred from the annular chamber 52 and into the inner chamber 50. The air transferred from the annular chamber 52 into the inner chamber 50 is replaced by air from the ambient environment which enters the annular chamber 52 via the lid openings 82.

When the user breathes out, the user blows air into the mouthpiece 18 which creates a positive pressure within the inner chamber 50. When the magnitude of the positive pressure within the inner chamber 50 is large enough, air is transferred from the inner chamber 50 to the annular chamber 52, and then vented out into the ambient environment through the lid openings 82.

If the user is too strained with breathing, the user may simply breathes through his/her nose or breathes normal without the mouthpiece 18. The goal is to breath in mouth and out mouth with resistance both on the inhale and exhale. The user should start slow and breathe at the user's own pace. Preferably, the user has a goal for a six count inhale, a two count hold, a six count exhale, and another two count hold, etc.

When a user begins the resistance breathing training, the user may start with just a few minutes a day for the first week. Eventually, a user may increase their training session to approximately fifteen minutes a day. With more serious health challenges, a user may build up to fifteen minutes, twice a day. After the user completes the resistance breathing session, it may be recommended to breathe in through the user's nose and out through the user's mouth.

Every inch of distance of air flow increases the subject amount of breathing and oxygen moved during the training session. Along these lines, the taller the inner and outer cups 14, 12 and longer the mouthpiece 18, the resistance is significantly enhanced. Furthermore, the resistance may be enhanced by adding water to the device. The extra water is another significant part of the resistance. The height of the device 10 also allows for a user of average height and up to 6′3″ to sit erect in a chair, open the lungs and breathe. Ideally, the user can sit upright, not looking down, and utilize the device 10. Adding more openings and/or smaller openings 82 on the top of the lid 16 adds increased resistance and the smaller holes do not allow the water to leak out the top of the cup on the exhale.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A resistance breathing device comprising:
a first body having a first internal chamber, the first body defining a primary opening, a secondary opening in fluid communication with the first internal chamber, and a breathing opening spaced from the primary and secondary openings and in fluid communication with the first internal chamber; and
an outer cup member having an open end portion and an opposed closed end portion, the outer cup member being sized and configured to be operatively connectable to the first body,
a portion of the first body being received within the outer cup member when the first body is connected to the outer cup member to define a second internal chamber between the first body and the outer cup member,
the second internal chamber being fluidly connectable to the first internal chamber via the secondary opening,
the outer cup member being configured to receive a liquid therein such that the secondary opening is submerged in the liquid to provide resistance to gas flowing between the first and second internal chambers.

2. The resistance breathing device recited in claim 1, wherein the first body includes an inner cup member having a closed end portion and an opposed open end portion, and a lid connectable to the open end portion of the inner cup member, the primary opening and the breathing opening being formed in the lid and the secondary opening being formed in the closed end portion of the inner cup member.

3. The resistance breathing device recited in claim 2, wherein:
the outer cup defines an inner diameter of approximately 2.75 inches and an internal length of approximately 5 inches;
the inner cup member defines an outer diameter of approximately 2 inches and an external length of approximately 4.875 inches.

4. The resistance breathing device recited in claim 1, wherein the second internal chamber circumscribes the first internal chamber.

5. The resistance breathing device recited in claim 1, wherein the outer cup member includes measurement lines formed on an inner surface thereof to denote liquid levels associated with respective levels of resistance.

6. The resistance breathing device recited in claim 1, further comprising a handle coupled to the outer cup member.

7. The resistance breathing device recited in claim 1, further comprising a straw connectable to the first body to fluidly connect the straw to the breathing opening.

8. The resistance breathing device recited in claim 7, further comprising a mouthpiece integral with the straw.

9. The resistance breathing device recited in claim 1, wherein the primary opening includes a plurality of primary holes and the secondary opening includes a plurality of secondary holes.

10. The resistance breathing device recited in claim 9, wherein the plurality of secondary holes defines at least two different shapes.

11. The resistance breathing device recited in claim 1, wherein the first body and the cup member are formed from a dishwasher safe material.

12. The resistance breathing device recited in claim 1, further comprising a metering device coupled to the first body and moveable relative thereto for selectively opening and closing the secondary opening.

13. The resistance breathing device recited in claim 12, wherein the metering device is rotatable relative to the first body.

14. A resistance breathing device comprising:
a main body having:
a first gas port,
a second gas port spaced from the first gas port, the main body defining a gas transfer passageway between the first and second gas ports; and
a reservoir within the gas transfer passageway, the reservoir being fillable with a liquid to provide resistance to gas transfer between the first and second gas ports,
the main body defining a longitudinal axis and being of an axial length that is approximately 5 inches.

15. The resistance breathing device recited in claim 14, wherein the gas transfer passageway includes a first gas chamber and a second gas chamber, the first and second gas chambers being separated by liquid in the reservoir when the reservoir is filled with liquid.

16. The resistance breathing device recited in claim 15, wherein the main body further includes a third gas port submersible within the liquid in the reservoir and configured to effectuate gas transfer between the first and second gas chambers.

17. The resistance breathing device recited in claim 16, further comprising a gas flow metering device moveably coupled to the main body and operative to selectively vary the size of the third gas port.

18. The resistance breathing device recited in claim 14, wherein the main body includes measurement lines formed on an inner surface thereof to denote liquid levels associated with respective levels of resistance.

19. The resistance breathing device recited in claim 14, further comprising a handle coupled to the main body.

20. The resistance breathing device recited in claim 14, further comprising a straw connectable to the main body adjacent one of the first and second gas ports.
